(19) United States
(12) Patent Application Publication
(10) Pub. No.: US 2024/0221154 A1
(43) Pub. Date: Jul. 4, 2024

(54) METHOD AND DEVICE FOR DISPLAYING BIO-IMAGE TISSUE

(71) Applicant: XAIMED CO., LTD, Seoul (KR)

(72) Inventor: Sang Min PARK, Seoul (KR)

(73) Assignee: XAIMED CO., LTD, Seoul (KR)

(21) Appl. No.: 18/288,804

(22) PCT Filed: Apr. 28, 2022
(86) PCT No.: PCT/KR2022/006063

§ 371 Date: Oct. 27, 2023

(30) Foreign Application Priority Data
Apr. 28, 2021 (KR) 10-2021-0055128

(51) Int. Cl. G06T 7/00; G06T 7/62; G06V 10/56; G06V 10/60

(52) U.S. Cl. CPC G06T 7/0012; G06T 7/62; G06V 10/56; G06V 10/60; G06T 2207/30096; G06T 2207/30204

(57) ABSTRACT

Provided is a device for displaying biological image tissue, comprising a processor and a memory including one or more instructions implemented to be performed by means of the processor, wherein the processor extracts information about a lesion from a first biological image obtained by photographing an object continuously over time on the basis of a machine learning model and processes the first biological image to generate a second biological image including a marker for displaying the information about the lesion, and a display unit for displaying the second biological image in a boundary region between an effective screen and an ineffective screen or in a region of the ineffective screen is included.

### TECHNICAL FIELD

The present disclosure relates to a method for displaying real-time biological images, and more specifically, to a method and apparatus for accurately displaying information about tissue in real-time biological images.

### BACKGROUND ART

As artificial intelligence learning models have developed, many machine learning models are being used to interpret images. For example, learning models such as convolutional neural networks (CNNs), deep neural networks (DNNs), recurrent neural networks (RNNs), and deep belief networks (DBNs) are applied to detection, classification, and feature learning of still images (Still Image) or real-time images (Motion Picture).

Although machine learning models are used to recognize and utilize attribute information in images (videos) as auxiliary materials for judgment, the display of attribute information does not take into account the user's working environment.

### DESCRIPTION OF EMBODIMENTS

### Technical Problem

The objective of the present disclosure is to provide a method and apparatus that can display a biological image tissue by visually and accurately recognizing attribute information in real-time images based on a machine learning model.

Another objective of the present disclosure is to provide a biological image tissue display method and apparatus that can visually secure sufficient field of view for attribute information in images.

The objectives of the present disclosure are not limited to the objectives mentioned above, and other objectives that are not mentioned can be clearly understood by a person of ordinary skill in the art from the following description.

### Solution to Problem

In one aspect of the present disclosure, an apparatus for displaying a tissue of a biological image comprises a processor, a memory that is communicatively coupled to the processor and stores one or more sequences of instructions, which when executed by the processor causes steps to be performed comprising: extracting a lesion information from a first biological image that has been continuously captured over time for a target object based on a machine learning model; and generating a second biological image including a marker for displaying the lesion information by image processing the first biological image, a display unit that displays the second biological image in a boundary area between a valid screen and an invalid screen or in an area of the invalid screen.

The lesion information may be 2-dimension or 3-dimension coordinates of the lesion within the valid screen of the display unit.

The lesion information may be a size of a lesion within the valid screen of the display unit.

The marker may be displayed at least two or more in at least one of the boundary areas between the valid screen and the invalid screen or the area of the invalid screen of the display unit.

The marker may be a first marker that indicates a location of a lesion.

The first marker may move depending on the movement of the lesion.

The marker may be a second marker that indicates a size of the lesion.

A size of the second marker may change depending on the size of the lesion.

The marker may be a third marker that indicates a presence or an absence of the lesion.

At least one of a brightness, color, or width of the third marker may change depending on the size of the lesion.

In another aspect of the present disclosure, A method for displaying a tissue of a biological image comprises extracting a lesion information from a first biological image that has been continuously captured over time for a target object based on a machine learning model; generating a second biological image including a marker for displaying the lesion information by image processing the first biological image; and displaying the second biological image in a boundary area between a valid screen and an invalid screen or in an area of the invalid screen.

The lesion information may be at least one of a presence, size, or location of the lesion.

### Advantageous Effects of Disclosure

According to the embodiments of the present disclosure, the tissue in real-time biological images can be visually accurately recognized based on a machine learning model.

In addition, according to the embodiments of the present disclosure, it can provide the user with visually sufficient surgical field of view for attribute information in images.

The effects of the present disclosure are not limited to the effects mentioned above, and other effects that are not mentioned can be clearly understood by a person of ordinary skill in the art from the following description.

### BRIEF DESCRIPTION OF DRAWINGS

FIG. 1 is a schematic diagram of an apparatus for biological image according to one embodiment of the present disclosure.

FIG. 2 is an illustrative diagram of the process of generating attribute information and images of real-time biological images by a computing device according to one embodiment of the present disclosure.

FIG. 3 is a block diagram of a processor for recognizing tissue in a biological image according to one embodiment of the present disclosure.

FIG. 4 is a biological image that has been processed by an apparatus according to one embodiment of the present disclosure.

FIG. 5 is a biological image generated in real time by an apparatus according to a first embodiment of the present disclosure.

FIG. 6 is a biological image generated in real time by an apparatus according to a second embodiment of the present disclosure.

FIG. 7 is a biological image generated in real time by an apparatus according to a third embodiment of the present disclosure.

FIG. 8 is a flow chart that illustrates an illustrative method for displaying tissue in biological images according to one embodiment of the present disclosure.

### DETAILED DESCRIPTION

The following, the embodiments of the present disclosure will be described in detail with reference to the attached drawings. However, it should be noted that the attached drawings are for the purpose of more easily disclosing the contents of the present invention, and that the scope of the present invention is not limited to the scope of the attached drawings. This will be easily understood by a person with ordinary knowledge in the relevant technical field.

In addition, the terms used in the detailed description and claims of the present disclosure are used only to describe a specific embodiment, and there is no intention to limit the invention. The singular expression includes the plural expression unless it is clearly intended to mean otherwise in the context.

In the detailed description and claims of the present disclosure, the terms “include” or “have” are understood to specify the existence of the features, numbers, steps, operations, components, parts, or combinations thereof described in the specification, but do not exclude the existence or addition of one or more other features or numbers, steps, operations, components, parts, or combinations thereof.

In the detailed description and claims of the present disclosure, the terms “learning” or “learning” are used to refer to the performance of machine learning through computing procedures, and are not intended to refer to mental activities such as human educational activities.

The term “real-time image data” used in the detailed description and claims of the present disclosure may be defined to include a single image (still image) or a series of images (video), and can be expressed as the same meaning as “image” or “image data”.

The term “image” used in the detailed description and claims of the present disclosure may be defined as a digital reproduction or imitation of the form or specific characteristics of a person or object, and the image may be a JPEG image, PNG image, GIF image, TIFF image, or any other digital image format known in the industry, but is not limited to that. Also, “image” can be used in the same sense as “photo”.

The term “attribute” used in the detailed description and claims of the present disclosure may be defined as a group of one or more descriptive characteristics of an object that can be recognized or detected within image data, and “attribute” can be expressed as a numerical characteristic.

The devices, methods, and devices disclosed in the present disclosure may be applied to any real-time biological tissue image that can support the diagnosis of medical images or disease states within the abdomen, but are not limited to that, and can be used for time-sequential computed tomography (CT), magnetic resonance imaging (MRI), computed radiography, magnetic resonance, vascular endoscopy, optical coherence tomography, color flow Doppler, cystoscopy, diaphanography, cardiac ultrasound, fluorescent angiography, laparoscopy, magnetic resonance angiography, positron emission tomography (PET), single photon emission computed tomography, X-ray angiography, nuclear medicine, biomagnetic imaging, colposcopy, duplex Doppler, digital microscopy, endoscopy, lasers, surface scanning, magnetic resonance spectroscopy, radiation graphic imaging, thermal imaging, and radiometric fluorescence imaging.

In addition, the present disclosure covers all possible combinations of embodiments shown in this specification. It should be understood that the various embodiments of the present disclosure are different, but need not be mutually exclusive. For example, the specific shape, structure, and characteristics described herein can be implemented as other embodiments without departing from the scope and concept of this invention in relation to a particular embodiment. In addition, the location or arrangement of individual components within each disclosed embodiment can be changed without departing from the scope and concept of the present disclosure. Therefore, the following detailed description is not intended to be taken in a restrictive sense, and the scope of this invention is limited only by the attached claims, as well as all ranges that are equivalent to what the claims claim if they are adequately described. Similar reference symbols in the drawings refer to the same or similar functions in multiple aspects.

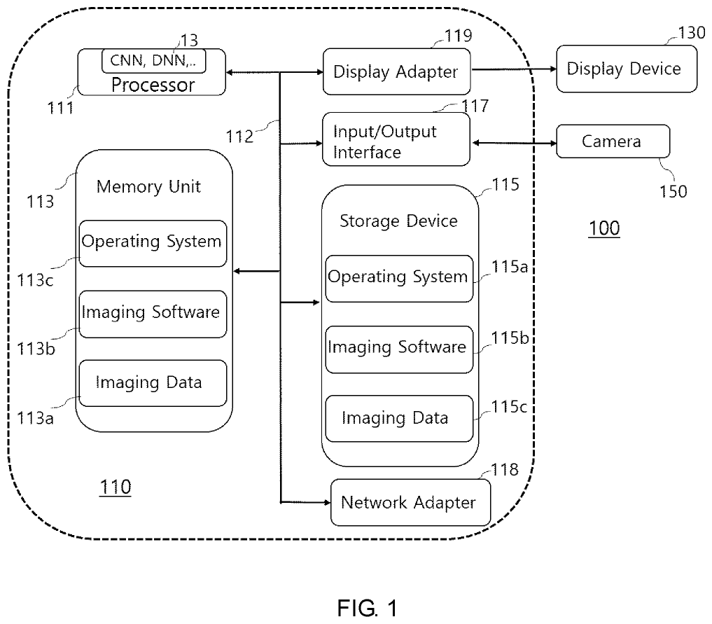

FIG. 1 is a schematic diagram of an apparatus for biological image according to one embodiment of the present disclosure.

Referring to FIG. 1, an apparatus (100) for displaying a real-time biological image tissue may include a computing device (110), a display device (130), and a camera (150). The computing device (110) may include a processor (111), a memory unit (113), a storage device (115), an input/output interface (117), a network adapter (118), a display adapter (119), and a system bus (112) connecting the processor to the memory unit (113), but is not limited to these. In addition, the apparatus may include other communication mechanisms in addition to the system bus (112) for transmitting information.

The system bus or other communication mechanisms connect the processor, the memory, which is a computer-readable recording medium, the near-field communication module (e.g., Bluetooth or NFC), the network adapter including the network interface or mobile communication module, the display device (e.g., CRT or LCD), the input device (e.g., keyboard, keypad, virtual keyboard, mouse, trackball, stylus, touch-sensitive means), and/or subsystems.

In one embodiment, the processor (111) may be a processing module that automatically processes using a machine learning model (13), and may be a CPU, AP (Application Processor), microcontroller, etc. that can process digital images, but is not limited to these.

In one embodiment, the processor (111) may communicate with a hardware controller for the display device, such as a display adapter (119), to display the operation and user interface of the tissue display device for biological images on the display device (130).

The processor (111) controls the operation of the apparatus according to the embodiments of the present disclosure to be described later by accessing the memory unit (113) and executing one or more sequences of instructions or logic stored in the memory unit.

These instructions may also be read from memory within a static storage or other computer-readable recording medium such as a disk drive. In other embodiments, hardware circuitry embedded in the hardware to replace or combine software instructions to implement the disclosure may also be used. Logic may also refer to any medium that participates in providing instructions to the processor and may be loaded into the memory unit (113).

In one embodiment, the system bus (112) represents one or more possible types of bus structures, including a memory bus or memory controller, a peripheral device bus, an accelerated graphics port, and a processor or local bus. For example, these architectures may include ISA (Industry Standard Architecture) bus, MCA (Micro Channel Architecture) bus, EISA (Enhanced ISA) bus, VESA (Video Electronics Standard Association) local bus, AGP (Accelerated Graphics Port) bus, and PCI (Peripheral Component Interconnects), PCI-Express bus, PCMCIA (Personal Computer Memory Card Industry Association), USB (Universal Serial Bus).

In one embodiment, the system bus (112) may be implemented as a wired or wireless network connection. Transmission media including the bus wires may include coaxial cables, copper wires, and optical fibers. In one example, the transmission media may take the form of sound waves or light waves generated during radio frequency communication or infrared data communication.

In one embodiment, the apparatus (100) may transmit and receive commands including messages, data, information, and one or more programs (i.e., application codes) through a network link and a network adapter (118). The network adapter (118) may also include a separate or integrated antenna to enable transmission and reception over a network link. The network adapter (118) may be connected to a network and communicate with a remote computing device (Remote Computing Device). The network may include LAN, WLAN, PSTN, and cellular phone networks, but is not limited to these.

In one embodiment, the network adapter (118) may include a network interface and a mobile communication module for connecting to the network. The mobile communication module is accessible to the generation network (for example, 2G to 5G mobile communication network).

The program code may be executed by the processor (111) when received, or stored in a non-volatile memory such as a disk drive in the memory unit (113) for execution.

In one embodiment, the computing device (110) may be a variety of computer-readable recording media. A readable medium may be any of a variety of media that can be accessed by a computing device, and may include, for example, volatile or non-volatile media, removable media, and non-removable media, but is not limited to these.

In one embodiment, the memory unit (113) may store the operating system, drivers, application programs, data, and database required for the operation of the biological image tissue recognition device according to the embodiments of the present invention, but is not limited to these. In addition, the memory unit (113) may include computer-readable media in the form of volatile memory such as RAM (Random Access Memory), read-only memory (ROM), and flash memory, and may also include disk drives such as hard disk drives (HDD), solid-state drives (SSD), and optical disc drives, but is not limited to these. In addition, the memory unit (113) and the storage device (115) typically include data such as imaging data (113a, 115a) such as biological images of the subject, program modules such as imaging software (113b, 115b) that can be immediately accessed by the processor (111), and operating systems (113c, 115c).

In one embodiment, the machine learning model (13) may be inserted in the processor (111), memory unit (113), or storage device (115). In this case, the machine learning model may include deep neural networks (DNN), convolutional neural networks (CNN), and recurrent neural networks (RNN), which are one of machine learning algorithms, but is not limited to these.

The camera unit (150) includes an image sensor that images the image of an object and converts the image into an image signal by photoelectric means, and captures the biological image of the subject in real time. A representative example is a biological image of the intestinal wall captured using an endoscope. The captured real-time biological image (image data) is provided to the processor (111) through the input/output interface (117) and processed based on the machine learning model (13) or stored in the memory unit (113) or storage device (115).

The apparatus for displaying biological images according to the present disclosure is not limited to laptop computers, desktop computers, and servers, and can be implemented in any computing device or system that can execute any command that can process data, and can be implemented in other computing devices and systems through the Internet network. In addition, the apparatus can be implemented in a variety of ways, including software, hardware, or a combination thereof, including firmware. For example, the function for performing in various ways can be performed by components that are implemented in various ways, including individual logic components, one or more ASICS (Application Specific Integrated Circuits), and/or program-controlled processors.

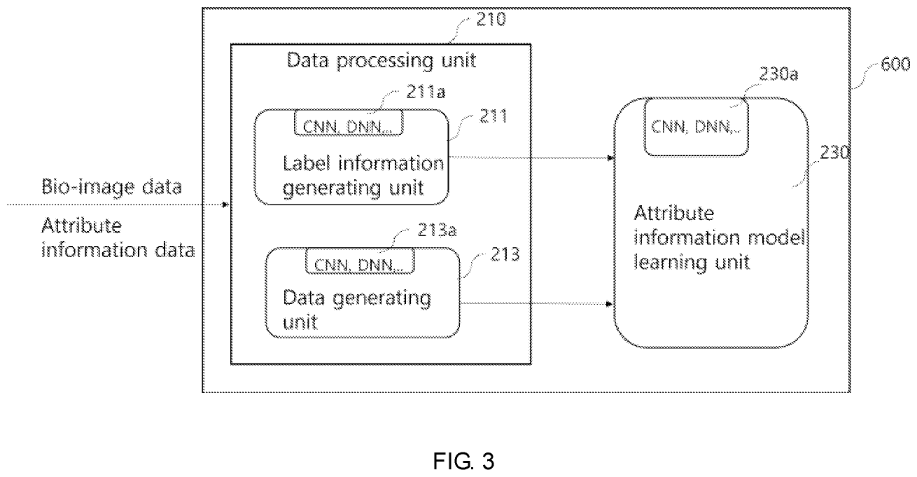

FIG. 3 is a block diagram of a processor for recognizing tissue in a biological image according to one embodiment of the present disclosure.

Referring to FIG. 3. The processor (600) may be the processor (111, 311) of FIG. 1, and may receive training data to train the machine learning model (211a, 213a, 215a, 230a), and may extract the property information of the training data based on the received training data. Training data may be real-time biological image data (multiple biological image data or single biological image data) or property information data extracted from real-time biological image data.

In one embodiment, the property information extracted from real-time biological image data may be label information that classifies the target detected in the biological image data. For example, the label may be a category classified as an organ such as liver, pancreas, and gallbladder expressed in the biological image data, a category classified as a tissue such as blood vessels, lymph, and nerves, and a category classified as a lesion of internal tissue such as fibroadenoma and tumor. In one embodiment, the label information may include the location information of the target (for example, the lesion), and the location information of the target may be expressed as a 2D coordinate (x, y) or a 3D coordinate (x, y, z). In addition, the label information may include the size information of the target (for example, the lesion), and the size information of the target may be expressed as width (Width) and height (Height). The label may be assigned a weight or order based on the weight, meaning of the target detected in the real-time biological image data.

The processor (600) may include a data processing unit (210) and a property information model learning unit (230).

The data processing unit (210) receives real-time biological image data and property information data for training the property information model (230), and transforms or processes the received biological image data and property information data into data suitable for the training of the property information model. The data processing unit (210) may include a label information generation unit (211), a data generation unit (213), and a feature extraction unit (215).

The label information generation unit (211) generates label information corresponding to the received real-time biological image data using the first machine learning model (211a). The label information may be information about one or more categories determined according to the target detected in the received real-time biological image data. In one embodiment, the label information may be stored in the memory unit (113) or storage device (115) along with information about the real-time biological image data corresponding to the label information.

The data generation unit (213) generates data to be input to the property information model learning unit (230) containing the machine learning model (230a). The data generation unit (213) uses the second machine learning model (213a) to generate input data to be input to the third machine learning model (230a) based on the multiple frame data included in the received real-time biological image data. Frame data may refer to each frame that composes a real-time biological image, RGB data for each frame that composes a real-time biological image, data that extracts features from each frame, or data that expresses features for each frame as a vector.

The property information model learning unit (230) includes the third machine learning model (230a), and extracts property information about the real-time biological image data by fusing learning (fusion learning) the data including the image data and label information generated and extracted from the label information generation unit (211) and the data generation unit (213). Property information refers to information related to the characteristics of the target image detected in the above real-time biological image data. For example, property information may be lesion information such as polyp, which classifies the target in the biological image data. If the property information extracted from the property information model learning unit is erroneous, the coefficients or connection weight values used in the third machine learning model (230a) can be updated.

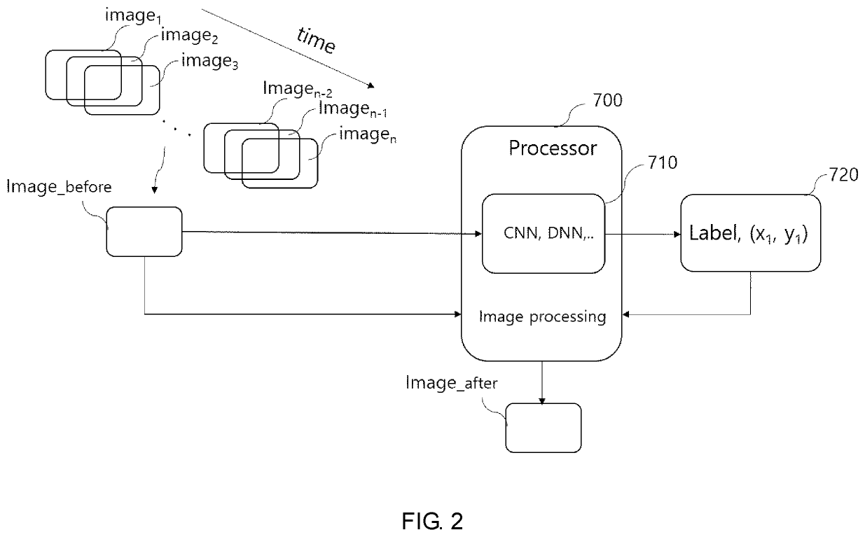

FIG. 2 is an illustrative diagram of the process of generating attribute information and images of real-time biological images by a computing device according to one embodiment of the present disclosure.

Referring to FIG. 2. a sequence of real-time biological images (image1, image2, . . . , imagen−1, imagen) captured in real time are input to the machine learning model (710), and the processor (700) extracts property information (720) of the input biological images (hereinafter referred to as the first biological image) based on the machine learning model (710) contained therein. Property information may be label information that classifies the target detected in the biological image, as described above, and label information may include location information of the target or size information of the target, etc. Property information (720) can be stored in the system memory unit (113) or storage device (115).

The processor (700) uses the extracted property information (720) to process the first biological image (Image_before) to generate the second biological image (Image_after). In this case, the second biological image may be processed by the processor (700) to include a marker to display the property information. The second biological image is displayed on the display unit under the control of the processor (700).

The machine learning model (710) is not shown, but can be input to a computer-readable recording medium and executed, and can be input to the memory unit (113) or storage device (115), and can be operated and executed by the above processor (700).

Such extraction of property information of real-time biological images can be performed by a computing device, and the computing device is a device that receives a dataset of real-time biological images as training data, and can generate learned data as a result of the execution of the machine learning model. In describing each operation belonging to the method according to the embodiment, if the description of the subject is omitted, the subject of the operation is understood to be the above computing device.

As shown in the above embodiment, it is clear that the operation and method of the present disclosure can be achieved by a combination of software and hardware or by hardware alone. The technical solutions of the present disclosure or the parts that contribute to the prior art can be implemented in the form of program instructions that can be executed through various computer components and recorded on a machine-readable recording medium. The above machine-readable recording medium may contain program instructions, data files, and data structures individually or in combination. The program instructions recorded on the above machine-readable recording medium may be specially designed and configured for the present disclosure, or they may be available for use by a person of ordinary skill in the art of computer software.

Examples of machine-readable recording media include magnetic media such as hard disks, floppy disks, and magnetic tapes, optical recording media such as CD-ROMs and DVDs, magneto-optical media such as floppy optical disks (floptical disks), and hardware devices specially configured to store and execute program instructions such as ROM, RAM, and flash memory. Examples of program instructions include machine code generated by a compiler, as well as high-level language code that can be executed by a computer using an interpreter, etc.

The above hardware device can be configured to operate as one or more software modules to perform processing according to the present disclosure, and vice versa. The above hardware device may include a processor such as a CPU or GPU that is configured to execute instructions stored in memory such as ROM/RAM for storing program instructions, and a communication unit that can exchange signals with external devices. In addition, the above hardware device may include external input devices such as a keyboard, mouse, and other external input devices to receive commands written by developers.

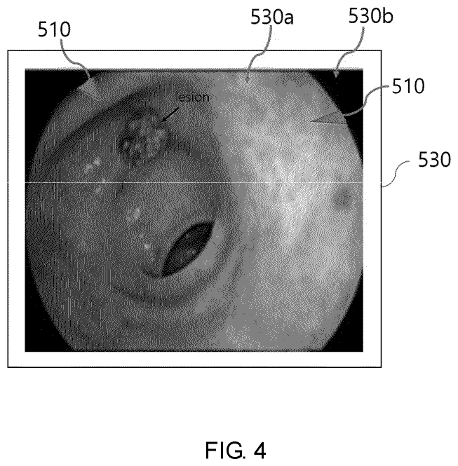

FIG. 4 is a biological image that has been processed by an apparatus according to one embodiment of the present disclosure.

Referring to FIG. 4. The display unit (530) where the biological image is displayed may include a valid screen (530a) and an invalid screen (530b). The valid screen (530a) is the part where the image of the target (e.g., tissue) is displayed, and the valid screen (530a) can be enlarged or reduced by the operation of the apparatus. The biological image displayed on the valid screen (530a) includes property information extracted based on the machine learning model, such as label information for lesion information, and a marker (510) to display the lesion.

The marker (510), as shown, can be displayed in the boundary area between the valid screen (530a) and the invalid screen (530b) of the display unit, but it can also be displayed in the area of the invalid screen (530b). In addition, the marker (510) can be displayed as a single unit, but it can be displayed as multiple units of at least two or more to accurately display the location information or size information of the lesion. In addition, the marker (510) can be displayed in a variety of shapes as long as it is a shape that can display lesion information.

Thus, the apparatus for displaying biological images according to the present disclosure, which displays an image including property information and a marker to display the property information, can accurately identify targets such as lesions in biological images to users (e.g., medical staff). In particular, the marker generated by the apparatus according to the present disclosure is not displayed on the valid screen where the lesion appears, so users can have a sufficient view of the lesion when performing various procedures such as biopsy, excision, and resection on the lesion, so they can perform the procedure stably.

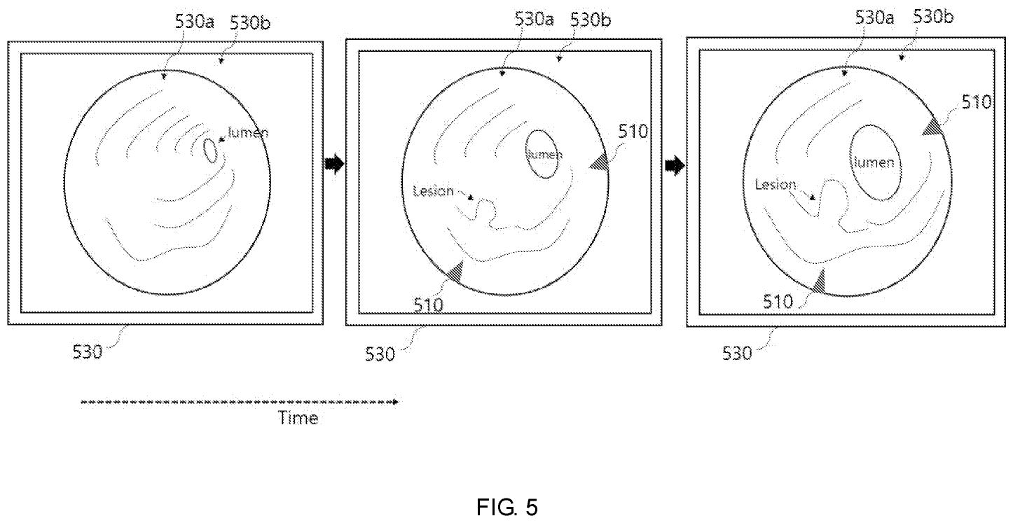

FIG. 5 is a biological image generated in real time by an apparatus according to a first embodiment of the present disclosure.

Referring to FIG. 5. The biological images generated on the display unit (530) are biological images generated over time while inspecting the internal tissue (e.g., stomach) of the human body using a device for biological images (e.g., an endoscope). The image of the tissue is displayed on the valid screen (530a) of the display unit (530), and when a lesion (Lesion) is recognized based on the machine learning model while moving the camera of the device, a first marker (510) to indicate the location of the lesion is displayed on the boundary area between the valid screen (530a) and the invalid screen (530b) of the display unit (530). At this time, the shape of the first marker (510) can be in a variety of shapes (e.g., an arrow) to indicate the location of the lesion, and the recognition of the lesion or the generation of the marker can be done in a variety of ways as described earlier. Also, the first marker (510) can be displayed in the area of the invalid screen (530b) of the display unit (530), which is not shown.

In one embodiment, the first marker (510) can be displayed at a different location on the boundary area between the valid screen (530a) and the invalid screen (530b) as the lesion's location changes with the movement of the camera. Also, the size of the first marker (510) may also change depending on the size of the lesion. The first marker (510) can be displayed as a single unit, but it can be displayed as multiple units of at least two or more to accurately identify the lesion's location.

The first marker (510) can be displayed in a different display method mode, and a guide line can be generated from the first marker (510) in the direction of the indication of the first marker (510). The intersection of the guide lines is the point where the lesion is located, so the user can more accurately identify the location of the lesion.

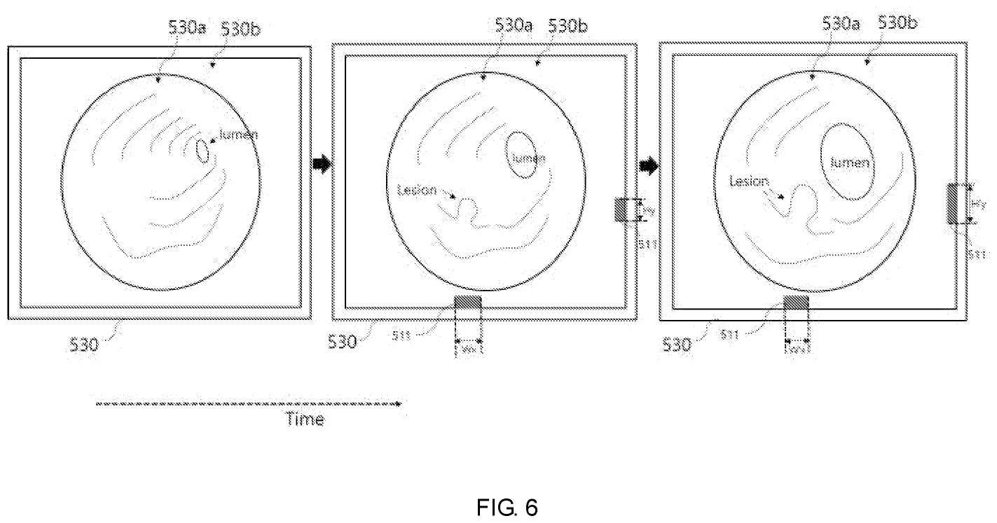

FIG. 6 is a biological image generated in real time by an apparatus according to a second embodiment of the present disclosure.

Refer to FIG. 6. The biological images generated on the display unit (530) are biological images generated over time while inspecting the internal tissue (e.g., stomach) of the human body using a tissue display device for biological images (e.g., an endoscope). The image of the tissue is displayed on the valid screen (530a) of the display unit (530), and when a lesion is recognized based on the machine learning model while moving the camera of the tissue display device for biological images, a second marker (511) to indicate the location of the lesion is displayed on the invalid screen (530b) area of the display unit (530). At this time, the shape of the second marker (511) can be in a variety of shapes (e.g., bar shape) to indicate the location and size of the lesion, and the recognition of the lesion or the generation of the marker can be done in a variety of ways as described earlier. The size of the lesion can be recognized by the size of the second marker (511), and the second marker (511) can be displayed in a size corresponding to the width (Wx) and height (Hy) of the lesion. Also, the second marker (511) can be displayed in the area of the valid screen (530a) and the invalid screen (530b) of the display unit (530), which is not shown.

In one embodiment, the second marker (511) can be displayed in a different size as the size of the lesion changes with the movement of the camera. Also, the second marker (511) can be displayed at a different location depending on the location of the lesion. The second marker (511) can be displayed as a single unit, but it can be displayed as multiple units of at least two or more to accurately identify the location or size of the lesion.

The second marker (511), which is not shown in the figure, can be displayed as a line extending from the second marker (511) in the horizontal and vertical directions based on the entire screen of the display unit (530) in a different display method mode. The intersection of these extension lines is the point where the lesion is located, so the user can more accurately identify the location and size of the lesion.

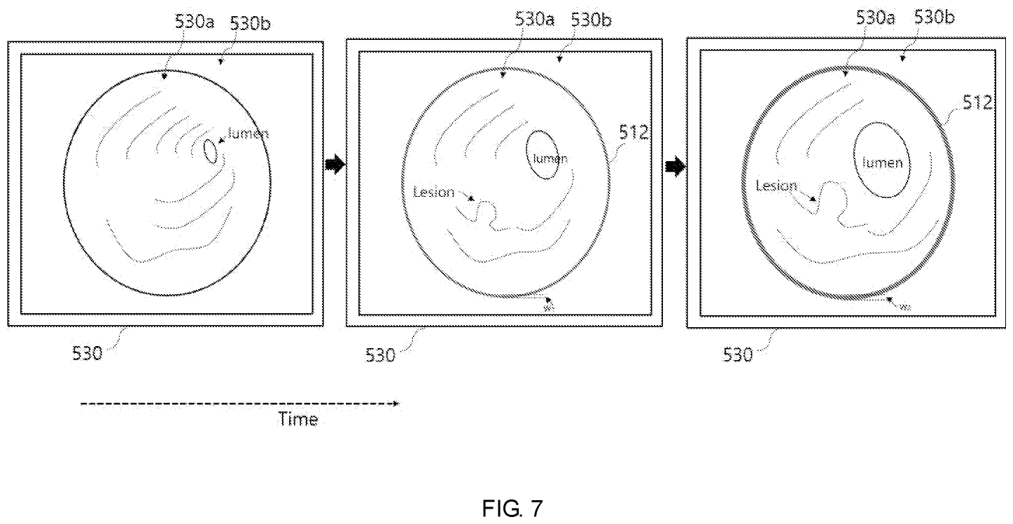

FIG. 7 is a biological image generated in real time by an apparatus according to a third embodiment of the present disclosure.

Referring to FIG. 7. The biological images generated on the display unit (530) are biological images generated over time while inspecting the internal tissue (e.g., stomach) of the human body using a device for biological images (e.g., an endoscope). The image of the tissue is displayed on the valid screen (530a) of the display unit (530), and when a lesion (Lesion) is recognized based on the machine learning model while moving the camera of the device, a third marker (512) to indicate the presence or absence of the lesion is displayed on the boundary area between the valid screen (530a) and the invalid screen (530b) of the display unit (530). At this time, the third marker (512) can be displayed throughout the boundary area where the image is displayed. The recognition of the lesion or the generation of the marker can be done in a variety of ways as described earlier.

In one embodiment, the third marker (512) can have its width (w1, w2) change depending on the size of the lesion as the camera moves. For example, the width of the third marker (512) can be larger as the size of the lesion increases. In addition, the brightness or color of the third marker (512) can change depending on the change in the size of the lesion. The brightness of the third marker (512) becomes brighter as the size of the lesion increases, and the color of the third marker (512) can have a larger color gradient level as the size of the lesion increases. In this way, the third marker (512) displayed in this manner can allow the user to accurately identify the presence or absence of a lesion in the image.

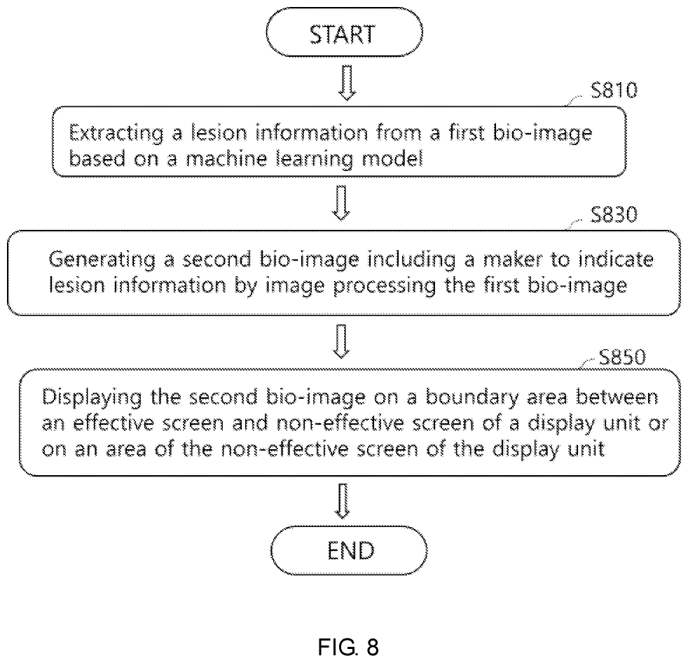

FIG. 8 is a flow chart that illustrates an illustrative method for displaying tissue in biological images according to one embodiment of the present disclosure.

Referring to FIG. 8. To extract the attribute information of the biological images (first bio-images) captured in real time, a machine learning model can be used. Attribute information can be information that can be labeled as categories such as organs or tissues in the biological image, but in this embodiment, we will explain the lesion information as an example. Machine learning models can include deep neural networks (DNN), convolutional neural networks (CNN), and recurrent neural networks (RNN), but are not limited to these.

In S810 step, the biological images (bio-image) captured in real time on the subject are input to the machine learning model, and the lesion information is extracted from the input biological images based on the machine learning model. Here, the biological images captured in real time can be a video image of the internal organs or tissues of the human body captured in real time using a camera such as a flexible endoscope or laparoscope, and in particular, any biological image of the internal organs of the human body captured in real time during surgery. Lesion information can include at least one of the existence, size, or location of the lesion, and the location of the lesion can be represented as 2D or 3D coordinates.

In S830 step, using the extracted lesion information, the biological images (bio-images) are processed by the processor of the apparatus of the present disclosure to generate a second biological image (a second bio-image). At this time, the second biological image can include a marker to indicate the lesion information. The marker can be displayed in a variety of shapes to indicate the existence, size, or location of the lesion, and can be displayed differently in color or brightness. In addition, the marker can be displayed with a different size depending on the size or location of the lesion.

Subsequently, in S850 step, the second biological image is displayed on the boundary area between the valid screen and invalid screen of the display unit or on the area of the invalid screen under the control of the processor. The valid screen is the part where the image of the target (e.g., tissue) is displayed, and the valid screen can be enlarged or reduced by zooming in or out by the operation of the biological image tissue display device.

As described above, it has been reviewed a desirable embodiment according to the present disclosure. It is self-evident to those with ordinary knowledge of the relevant technology that the present invention can be concretized in other specific forms without deviating from its purpose or category, in addition to the embodiments described above. Therefore, the aforementioned embodiments should be considered to be illustrative rather than restrictive, and accordingly, the present invention may be modified within the scope of the attached claim and its equivalent range without being limited to the explanation mentioned above.

1. An apparatus for displaying a tissue of a biological image comprising:
a processor;
a memory that is communicatively coupled to the processor and stores one or more sequences of instructions, which when executed by the processor causes steps to be performed comprising:
extracting a lesion information from a first biological image that has been continuously captured over time for a target object based on a machine learning model; and
generating a second biological image including a marker for displaying the lesion information by image processing the first biological image,
a display unit that displays the second biological image in a boundary area between a valid screen and an invalid screen or in an area of the invalid screen.

2. The apparatus of claim 1,
wherein the lesion information is 2-dimension or 3-dimension coordinates of the lesion within the valid screen of the display unit.

3. The apparatus of claim 1,
wherein the lesion information is a size of a lesion within the valid screen of the display unit.

4. The apparatus of claim 1,
wherein the marker is displayed at least two or more in at least one of the boundary areas between the valid screen and the invalid screen or the area of the invalid screen of the display unit.

5. The apparatus of claim 1,
wherein the marker is a first marker that indicates a location of a lesion.

6. The apparatus of claim 5,
wherein the first marker moves depending on the movement of the lesion.

7. The apparatus of claim 1,
wherein the marker is a second marker that indicates a size of the lesion.

8. The apparatus of claim 1,
wherein a size of the second marker changes depending on the size of the lesion.

9. The apparatus of claim 1,
wherein the marker is a third marker that indicates a presence or an absence of the lesion.

10. The apparatus of claim 9,
wherein at least one of a brightness, color, or width of the third marker changes depending on the size of the lesion.

11. A method for displaying a tissue of a biological image, comprising:
extracting a lesion information from a first biological image that has been continuously captured over time for a target object based on a machine learning model;
generating a second biological image including a marker for displaying the lesion information by image processing the first biological image; and
displaying the second biological image in a boundary area between a valid screen and an invalid screen or in an area of the invalid screen.

12. The method of claim 11,
wherein the lesion information is at least one of a presence, size, or location of the lesion.
